(19) United States
(12) Patent Application Publication
(10) Pub. No.: US 2022/0071868 A1
(43) Pub. Date: Mar. 10, 2022

(54) Methods of Inhibiting Neutrophil Recruitment to the Gingival Crevice

(71) Applicant: Colgate-Palmolive Company, New York, NY (US)

(72) Inventors: Camille ZENOBIA, Hampton, NJ (US); Carlo DAEP, Brooklyn, NY (US); Dandan CHEN, Beridgewater, NJ (US); Harsh Mahendra TRIVEDI, Hillsborough, NJ (US); James MASTERS, Ringoes, NJ (US)

(73) Assignee: Colgate-Palmolive Company, New York, NY (US)

(21) Appl. No.: 17/309,895

(22) PCT Filed: Dec. 17, 2019
(86) PCT No.: PCT/US2019/066834

§ 371 Date: Jun. 28, 2021

Related U.S. Application Data
(60) Provisional application No. 62/785,147, filed on Dec. 26, 2018.

(51) Int. Cl. A61K 8/27; A61K 8/21

(52) U.S. Cl. CPC A61K 8/27; A61Q 11/00; A61K 8/21

(57) ABSTRACT

Methods of increasing proteins that reduce signals associated with neutrophil recruitment and reducing proteins that induce neutrophil recruitment within an individuals gingival crevice are disclosed. The methods comprise applying to the individual's oral cavity in an amount effective to increase proteins that reduce signals associated with neutrophil recruitment and reduce proteins that induce neutrophil recruitment within an individuals gingival crevice, an oral care composition comprising: zinc oxide, zinc citrate, stannous fluoride, and optionally arginine and/or zingerone.

### BACKGROUND

The gums, also referred to as gingiva, are a part of the soft tissue lining of the mouth that surround the teeth and provide a seal around them. The gingival margin is the interface between the sulcular epithelium and the epithelium of the oral cavity. This interface exists at the most coronal point of the gingiva, otherwise known as the crest of the marginal gingiva. The gingival crevice, also called gingival sulcus, is the space located around a tooth between the wall of the unattached gum tissue and the enamel and/or cementum of the tooth.

Gum disease is an inflammation of the gum line that can progress to affect the bone that surrounds and supports your teeth. The three stages of gum disease—from least to most severe—are gingivitis, periodontitis and advanced periodontitis.

Gingivitis is the initial stage of gum disease. The direct cause of gingivitis is plaque—the soft, sticky, colorless film of bacteria that forms constantly on the teeth and gums. If the plaque is not removed by daily brushing and flossing, accumulates and hardens over time. The bacteria found in this buildup produces toxins that can irritate the gum tissue, causing gingivitis.

Gingivitis is treatable. Damage can be reversed, since the bone and connective tissue that hold the teeth in place are not yet affected. Left untreated, however, gingivitis can become an advanced stage of gum disease, periodontitis, and cause permanent damage to teeth and jaw.

The bacteria produced by the plaque irritate the gums, triggering the immune system to produce powerful bacteria-fighting elements that attack the infection. An unfortunate consequence is that these elements inadvertently destroy bone and tissue responsible for supporting the teeth. Essentially the body turns on itself. As tissue is broken down, spaces begin to form separating the gums from the teeth. These spaces become infected and deepen, further destroying gum tissue and bone. Eventually when there is an insufficient amount of bone left to support teeth, they begin to feel loose and may have to be removed.

Neutrophils, which are a type of granulocyte, are an abundant type of white blood cells that form an essential part of the innate immune system. During the beginning phase of inflammation in the gums, neutrophils are one of the first-responders of inflammatory cells to migrate towards the site of inflammation. The migration of neutrophils is regulated by various chemical signals in a process called chemotaxis.

Examples of proteins that can reduce neutrophil recruitment include IL-1Ra and sICAM, and examples of proteins that induce neutrophil recruitment are selected from the group consisting of: TNF-a, RANTES, MIF, GCSF, CXCL10 and GRO. Increasing levels of proteins that reduce neutrophil recruitment such as IL-1Ra and sICAM in the gingival crevice have an anti-inflammatory effect that reduces the negative consequences of inflammation of the gums. Likewise, reducing levels of proteins that promote neutrophil recruitment such as TNF-a, RANTES, MIF, GCSF, CXCL10 and GRO in the gingival crevice have an anti-inflammatory effect that reduces the negative consequences of inflammation of the gums.

### BRIEF SUMMARY

Methods of controlling inflammation of the gums and promoting healthy gums and overall good oral health are provided.

Methods of increasing proteins that reduce signals associated with neutrophil recruitment and reducing proteins that induce neutrophil recruitment within an individual's gingival crevice are provided. The methods comprise applying to the individual's oral cavity in an amount effective to increase proteins that reduce signals associated with neutrophil recruitment and reduce proteins that induce neutrophil recruitment within an individual's gingival crevice, an oral care composition comprising: zinc oxide, zinc citrate, stannous fluoride, and optionally arginine and/or zingerone.

### BRIEF DESCRIPTION OF THE DRAWINGS

FIG. 1 is an illustration of a GCM.

FIG. 2 shows data from GCM experiments comparing IL-1RA levels in response to treatment with various formulations.

FIG. 3 shows data from GCM experiments comparing CXCL10 levels in response to treatment with various formulations.

FIG. 4 shows data from GCM experiments comparing ICAM-1 levels in response to treatment with various formulations.

FIG. 5 shows data from GCM experiments comparing G-CSF levels in response to treatment with various formulations.

FIG. 6 shows data from GCM experiments comparing TNFa levels in response to treatment with various formulations.

FIG. 7 shows data from GCM experiments comparing RANTES levels in response to treatment with various formulations.

### DETAILED DESCRIPTION

Application of oral care compositions to the oral cavity of an individual can affect the inflammatory signals in the individual's gingival crevice including the reduction of pro-inflammatory signals and the upregulation of signals that block inflammation. Reducing signals which recruit neutrophils and promoting signals that reduce neutrophil recruitment provides effective strategies to control inflammation and promote healthy gums and overall good oral health.

A model has been designed for testing biological efficacy of oral health compounds. The model employs a unique combination of cells and bacterial biofilm in an in vitro cell culture that allows for the measure of inflammatory biomarkers that are predictive of clinical effects. The model is helpful in predicting product efficacy.

The model, which is referred to as a gingival crevice model (GCM), includes layered primary gingival epithelial cells, such as tissue commercially available from MatTek), coupled with neutrophil-like cells that are generated by inducing HL60 cells (ATCC) to a neutrophil like phenotype with retinoic acid. The model simulates what is seen morphologically within healthy junctional gingival tissues. An ex vivo derived biofilm, generated from saliva donation and created on substrates, such as HAP, poly-D-lysine, collagen-coated or enamel disks, collagen matrices, and polydimethylsiloxane (PDMS), agarose, agar, poly(ethylene glycol) dimethacrylate (PEGDMA) and 2-methacryloyloxyethyl phosphorylcholine polymer (PMPC) hydrogels is added to the epithelial cell layer. To simulate an inflammatory disease-like state within the model system, Fetal Bovine serum may be added. The model allows for rapid analysis of oral care products such as toothpaste, mouthwash, etc.

The GCM is useful to test a compound or formulation's ability to prevent or resolve inflammation. The GCM is also useful to test the compound or formulation's effect on oral bacteria and biofilm, which are generated by saliva donation and cultivation on substrates, such as HAP, poly-D-lysine, collagen-coated, enamel disks or on “soft” substrates such as, for example, substrates made from collagen matrices such as CollaForm® Collagen Wound Dressing material (Impladent Ltd., Jamaica, N.Y.), or substrates made from polydimethylsiloxane (PDMS), agarose, agar, poly(ethylene glycol) dimethacrylate (PEGDMA), and 2-methacryloyloxyethyl phosphorylcholine polymer (PMPC) hydrogels, to predict health or disease status. The model provides predicative clinical measures.

Using the GCM, formulations were tested and analyzed for effects in chemokine/cytokine production. Oral care compositions, particularly tooth pastes that comprise zinc oxide, zinc citrate and stannous fluoride, and optionally arginine or zingerone, were found to reduce signals associated with neutrophil recruitment and a promote signals that reduce neutrophil recruitment. Proteins that reduce signals associated with neutrophil recruitment include IL-1RA and sICAM-1. Proteins that promote neutrophil recruitment include: TNF-a, RANTES (during inflammation), MIF, GCSF, CXCL10 and GRO. By reducing signals associated with neutrophil recruitment and promoting signals that reduce neutrophil recruitment, the oral care compositions are useful as compositions that control inflammation and promote healthy gums and overall good oral health.

Embodiments provided herein include methods that comprise applying to the gingival crevice in the oral cavity of an individual an oral care composition that comprises an effective amount of zinc oxide, zinc citrate, stannous fluoride and optionally arginine and/or zingerone. In some embodiments, oral care compositions are a toothpaste or a mouthwash.

In some embodiments the oral care compositions comprise zinc oxide to zinc citrate in a ratio from 1.5:1 to 4.5:1, 1.5:1 to 4:1, 1.7:1 to 2.3:1, 1.9:1 to 2.1:1, or about 2:1. Also, the corresponding molar ratios based on these weight ratios can be used. In some embodiments, the total concentration of zinc salts in the composition is from 0.2 weight % to 5 weight %, or from 0.5 weight % to 2.5 weight % or from 0.8 weight % to 2 weight %, or about 1.5 weight %, based on the total weight of the composition. In some embodiments, the molar ratio of arginine to total zinc salts is from 0.05:1 to 10:1. In some embodiments, the composition comprises zinc oxide in an amount of from 0.5 weight % to 1.5 weight % and zinc citrate in an amount of from 0.25 weight % to 0.75 weight %, based on the total weight of the composition. In some embodiments, the composition may comprise zinc oxide in an amount of from 0.75 weight % to 1.25 weight % and zinc citrate in an amount of from 0.4 weight % to 0.6 weight %, based on the total weight of the composition. In some embodiments, the composition comprises zinc oxide in an amount of about 1 weight % and zinc citrate in an amount of about 0.5 weight %, based on the total weight of the composition. In some embodiments, zinc oxide may be present in an amount of from 0.75 to 1.25 wt % (e.g., 1.0 wt. %) the zinc citrate is in an amount of from 0.25 to 1.0 wt % (e.g. 0.25 to 0.75 wt. %, or 0.5 wt. %) and based on the weight of the oral care composition. In some embodiments, the zinc citrate is about 0.5 wt %. In some embodiments, the zinc oxide is about 1.0 wt %.

In some embodiments the ZnO particles may have an average particle size of from 1 to 7 microns. In some embodiments, the ZnO particles have an average particle size of 5 microns or less. In some embodiments, suitable zinc oxide particles for oral care compositions have, for example, a particle size distribution of 3 to 4 microns, or alternatively, a particle size distribution of 5 to 7 microns, alternatively, a particle size distribution of 3 to 5 microns, alternatively, a particle size distribution of 2 to 5 microns, or alternatively, a particle size distribution of 2 to 4 microns. Zinc oxide may have a particle size which is a median particle size. Suitable particles may have, for example, a median particle size of 8 microns or less, alternatively, a median particle size of 3 to 4 microns, alternatively, a median particle size of 5 to 7 microns, alternatively, a median particle size of 3 to 5 microns, alternatively, a median particle size of 2 to 5 microns, or alternatively, a median particle size of 2 to 4 microns. In another aspect, that particle size is an average (mean) particle size. In an embodiment, the mean particle comprises at least 5%, at least 10%, at least 15%, at least 20%, at least 25%, at least 30%, at least 35%, or at least 40% of the total metal oxide particles in an oral care composition of the invention. The particle may be present in an amount of up to 5% by weight, based on the total weight of the oral care composition, for example in an amount of from 0.5 to 5% by weight, preferably of up to 2% by weight, more preferably from 0.5 to 2% by weight, more preferably from 1 to 2% by weight, or in some embodiment from 2.5 to 4.5% by weight, being based on the total weight of the oral care composition. In some embodiments, the source of zinc oxide particles and/or the form they may be incorporated into the oral care composition in is selected from one or more of a powder, a nanoparticle solution or suspension, or encapsulated in a polymer or bead. Zinc oxide particles may be selected to achieve occlusion of dentin particles. Particle size distribution may be measured using a Malvern Particle Size Analyzer, Model Mastersizer 2000 (or comparable model) (Malvern Instruments, Inc., Southborough, Mass.), wherein a helium-neon gas laser beam is projected through a transparent cell which contains silica, such as, for example, silica hydrogel particles suspended in an aqueous solution. Light rays which strike the particles are scattered through angles which are inversely proportional to the particle size. The photodetector arrant measures the quantity of light at several predetermined angles. Electrical signals proportional to the measured light flux values are then processed by a microcomputer system, against a scatter pattern predicted from theoretical particles as defined by the refractive indices of the sample and aqueous dispersant to determine the particle size distribution of the metal oxide. It will be understood that other methods of measuring particle size are known in the art, and based on the disclosure set forth herein, the skilled artisan will understand how to calculate median particle size, mean particle size, and/or particle size distribution of metal oxide particles.

Stannous fluoride is present in a clinically efficacious amount. Fluoride where present may be present at levels of, e.g., about 25 to about 25,000 ppm, for example about 50 to about 5000 ppm, about 750 to about 2,000 ppm for a consumer toothpaste (e.g., 1000-1500 ppm, e.g., about 1000 ppm, e.g., about 1450 ppm). In some embodiments, fluoride is present from about 100 to about 1000, from about 200 to about 500, or about 250 ppm fluoride ion. 500 to 3000 ppm. In some embodiments, the fluoride source provides fluoride ion in an amount of from 50 to 25,000 ppm (e.g., 750-7000 ppm, e.g., 1000-5500 ppm, e.g., about 500 ppm, 1000 ppm, 1100 ppm, 2800 ppm, 5000 ppm, or 25000 ppm). In some embodiments, the fluoride source is stannous fluoride which provides fluoride in an amount from 750-7000 ppm (e.g., about 1000 ppm, 1100 ppm, 2800 ppm, 5000 ppm). In some embodiments, the fluoride source is stannous fluoride which provides fluoride in an amount of about 5000 ppm. Fluoride ion sources may be added to the compositions at a level of about 0.001 wt. % to about 10 wt. %, e.g., from about 0.003 wt. % to about 5 wt. %, 0.01 wt. % to about 1 wt., or about 0.05 wt. %. In some embodiment, the stannous fluoride is present in an amount of 0.1 wt. % to 2 wt. % (0.1 wt %-0.6 wt. %) of the total composition weight.

Oral care compositions optionally comprise arginine or a salt thereof. In some embodiments, the arginine is L-arginine or a salt thereof. Suitable salts include salts known in the art to be pharmaceutically acceptable salts are generally considered to be physiologically acceptable in the amounts and concentrations provided. Physiologically acceptable salts include those derived from pharmaceutically acceptable inorganic or organic acids or bases, for example acid addition salts formed by acids which form a physiological acceptable anion, e.g., hydrochloride or bromide salt, and base addition salts formed by bases which form a physiologically acceptable cation, for example those derived from alkali metals such as potassium and sodium or alkaline earth metals such as calcium and magnesium. Physiologically acceptable salts may be obtained using standard procedures known in the art, for example, by reacting a sufficiently basic compound such as an amine with a suitable acid affording a physiologically acceptable anion. In some embodiments, the arginine in partially or wholly in salt form such as arginine phosphate, arginine hydrochloride or arginine bicarbonate. In some embodiments, the arginine is present in an amount corresponding to 0.1% to 15%, e.g., 0.1 wt % to 10 wt %, e.g., 0.1 to 5 wt %, e.g., 0.5 wt % to 3 wt % of the total composition weight, about e.g., 1%, 1.5%, 2%, 3%, 4%, 5%, or 8%, wherein the weight of the arginine is calculated as free form. In some embodiments the arginine is present in an amount corresponding to about 0.5 wt. % to about 20 wt. % of the total composition weight, about 0.5 wt. % to about 10 wt. % of the total composition weight, for example about 1.5 wt. %, about 3.75 wt. %, about 5 wt. %, or about 7.5 wt. % wherein the weight of the arginine is calculated as free form. In some embodiments, the arginine is present in an amount of from 0.5 weight % to 10 weight %, or from 0.5 weight % to 3 weight % or from 1 weight % to 2.85 weight %, or from 1.17 weight % to 2.25 weight %, based or from 1.4 weight % to 1.6 weight %, or from 0.75 weight % to 2.9 weight %, or from 1.3 weight % to 2 weight %, or about 1.5 weight %, based on the total weight of the composition. Typically, the arginine is present in an amount of up to 5% by weight, further optionally from 0.5 to 5% by weight, still further optionally from 2.5 to 4.5% by weight, based on the total weight of the oral care composition. In some embodiments, arginine is present in an amount from 0.1 wt. %-6.0 wt. %. (e.g., about 1.5 wt %) or from about 4.5 wt. %-8.5 wt. % (e.g., 5.0%) or from 3.5 wt. %-9 wt. % or 8.0 wt. %. In some embodiments, the arginine is present in a dentifrice, at for example about 0.5-2 wt. %, e.g., and about 0.8% in the case of a mouthwash.

The oral care compositions optionally comprise zingerone. In some embodiments, the zingerone is present in an amount of from 0.01% to 1% (e.g., 0.05% to 0.5%; e.g., 0.05% to 0.35%; e.g., 0.1%, 0.2%, or 0.3%),

The oral care compositions described herein may also comprise one or more further agents such as those typically selected from the group consisting of: abrasives, an anti-plaque agent, a whitening agent, antibacterial agent, cleaning agent, a flavoring agent, a sweetening agent, adhesion agents, surfactants, foam modulators, pH modifying agents, humectants, mouth-feel agents, colorants, tartar control (anti-calculus) agent, polymers, saliva stimulating agent, nutrient, viscosity modifier, anti-sensitivity agent, antioxidant, and combinations thereof.

In some embodiments, the oral care compositions comprise one or more abrasive particulates such as those useful for example as a polishing agent. Any orally acceptable abrasive can be used, but type, fineness, (particle size) and amount of abrasive should be selected so that tooth enamel is not excessively abraded in normal use of the composition. Examples of abrasive particulates may be used include abrasives such sodium bicarbonate, insoluble phosphates (such as orthophosphates, polymetaphosphates and pyrophosphates including dicalcium orthophosphate dihydrate, calcium pyrophosphate, tricalcium phosphate, calcium polymetaphosphate and insoluble sodium polymetaphosphate), calcium phosphate (e.g., dicalcium phosphate dihydrate), calcium sulfate, natural calcium carbonate (CC), precipitated calcium carbonate (PCC), silica (e.g., hydrated silica or silica gels or in the form of precipitated silica or as admixed with alumina), iron oxide, aluminium oxide, aluminum silicate, calcined alumina, bentonite, other siliceous materials, perlite, plastic particles, e.g., polyethylene, and combinations thereof. The natural calcium carbonate abrasive of is typically a finely ground limestone which may optionally be refined or partially refined to remove impurities. The material preferably has an average particle size of less than 10 microns, e.g., 3-7 microns, e.g. about 5.5 microns. For example, a small particle silica may have an average particle size (D50) of 2.5-4.5 microns. Because natural calcium carbonate may contain a high proportion of relatively large particles of not carefully controlled, which may unacceptably increase the abrasivity, preferably no more than 0.01%, preferably no more than 0.004%) by weight of particles would not pass through a 325 mesh. The material has strong crystal structure, and is thus much harder and more abrasive than precipitated calcium carbonate. The tap density for the natural calcium carbonate is for example between 1 and 1.5 g/cc, e.g., about 1.2 for example about 1.19 g/cc. There are different polymorphs of natural calcium carbonate, e.g., calcite, aragonite and vaterite, calcite being preferred for purposes of this invention. An example of a commercially available product suitable for use in the present invention includes Vicron® 25-11 FG from GMZ. Precipitated calcium carbonate has a different crystal structure from natural calcium carbonate. It is generally more friable and more porous, thus having lower abrasivity and higher water absorption. For use in the present invention, the particles are small, e.g., having an average particle size of 1-5 microns, and e.g., no more than 0.1%, preferably no more than 0.05% by weight of particles which would not pass through a 325 mesh. The particles may for example have a D50 of 3-6 microns, for example 3.8-4.9, e.g., about 4.3; a D50 of 1-4 microns, e.g. 2.2-2.6 microns, e.g., about 2.4 microns, and a D10 of 1-2 microns, e.g., 1.2-1.4, e.g. about 1.3 microns. The particles have relatively high water absorption, e.g., at least 25 g/100 g, e.g. 30-70 g/100 g. Examples of commercially available products suitable for use include, for example, Carbolag® 15 Plus from Lagos Industria Quimica. In some embodiments, additional calcium-containing abrasives, for example calcium phosphate abrasive, e.g., tricalcium phosphate, hydroxyapatite or dicalcium phosphate dihydrate or calcium pyrophosphate, and/or silica abrasives, sodium metaphosphate, potassium metaphosphate, aluminum silicate, calcined alumina, bentonite or other siliceous materials, or combinations thereof are used. Examples of silica abrasives include, but are not limited to, precipitated or hydrated silicas having a mean particle size of up to about 20 microns (such as Zeodent 105 and Zeodent 1 14 marketed by J.M. Huber Chemicals Division, Havre de Grace, Md. 21078); Sylodent 783 (marketed by Davison Chemical Division of W.R. Grace & Company); or Sorbosil AC 43 (from PQ Corporation). In some embodiments, an effective amount of a silica abrasive is about 10-30%, e.g. about 20%. In some embodiments, the acidic silica abrasive Sylodent is included at a concentration of about 2 to about 35% by weight; about 3 to about 20% by weight, about 3 to about 15% by weight, about 10 to about 15% by weight. For example, the acidic silica abrasive may be present in an amount selected from 2 wt. %, 3wt. %, 4% wt. %, 5 wt. %, 6 wt. %, 7 wt. %, 8 wt. %, 9 wt. %, 10 wt. %, 11 wt. %, 12 wt. %, 13 wt. %, 14 wt. %,15 wt. %, 16 wt. %, 17 wt. %, 18 wt. %, 19 wt. %, 20 wt. %. Sylodent 783 has a pH of 3.4-4.2 when measured as a 5% by weight slurry in water and silica material has an average particle size of less than 10 microns, e.g., 3-7 microns, e.g. about 5.5 microns. In some embodiments, the silica is synthetic amorphous silica, (e.g., 1%-28% by wt.) (e.g., 8%-25% by wt). In some embodiments, the silica abrasives are silica gels or precipitated amorphous silicas, e.g. silicas having an average particle size ranging from 2.5 microns to 12 microns. Some embodiments further comprise a small particle silica having a median particle size (d50) of 1-5 microns (e.g., 3-4 microns) (e.g., about 5 wt. % Sorbosil AC43 from PQ Corporation Warrington, United Kingdom). The composition may contain from 5 to 20 wt % small particle silica, or for example 10-15 wt %, or for example 5 wt %, 10 wt %, 15 wt % or 20 wt % small particle silica. In some embodiments, 20-30 wt % of the total silica in the composition is small particle silica (e.g., having a median particle size (d50) of 3-4 microns and wherein the small particle silica is about 5 wt. % of the oral care composition. In some embodiments, silica is used as a thickening agent, e.g., particle silica. In some embodiments, the composition comprises calcium carbonate, such as precipitated calcium carbonate high absorption (e.g., 20% to 30% by weight of the composition or, 25% precipitated calcium carbonate high absorption), or precipitated calcium carbonate-light (e.g., about 10% precipitated calcium carbonate-light) or about 10% natural calcium carbonate.

In some embodiments, the oral care compositions comprise a whitening agent, e.g., a selected from the group consisting of peroxides, metal chlorites, perborates, percarbonates, peroxyacids, hypochlorites, hydroxyapatite, and combinations thereof. Oral care compositions may comprise hydrogen peroxide or a hydrogen peroxide source, e.g., urea peroxide or a peroxide salt or complex (e.g., such as peroxyphosphate, peroxycarbonate, perborate, peroxysilicate, or persulphate salts; for example, calcium peroxyphosphate, sodium perborate, sodium carbonate peroxide, sodium peroxyphosphate, and potassium persulfate or hydrogen peroxide polymer complexes such as hydrogen peroxide-polyvinyl pyrrolidone polymer complexes.

In some embodiments, the oral care compositions comprise an effective amount of one or more antibacterial agents, for example comprising an antibacterial agent selected from halogenated diphenyl ether (e.g. triclosan), triclosan monophosphate, herbal extracts and essential oils (e.g., rosemary extract, tea extract, magnolia extract, thymol, menthol, eucalyptol, geraniol, carvacrol, citral, hinokitol, magonol, ursolic acid, ursic acid, morin, catechol, methyl salicylate, epigallocatechin gallate, epigallocatechin, gallic acid, miswak extract, sea-buckthorn extract), bisguanide antiseptics (e.g., chlorhexidine, alexidine or octenidine), quaternary ammonium compounds (e.g., cetylpyridinium chloride (CPC), benzalkonium chloride, tetradecylpyridinium chloride (TPC), N-tetradecyl-4-ethylpyridinium chloride (TDEPC)), phenolic antiseptics, hexetidine furanones, bacteriocins, ethyllauroyl arginate, arginine bicarbonate, a Camellia extract, a flavonoid, a flavan, halogenated diphenyl ether, creatine, sanguinarine, povidone iodine, delmopinol, salifluor, metal ions (e.g., zinc salts, stannous salts, copper salts, iron salts), propolis and oxygenating agents (e.g., hydrogen peroxide, buffered sodium peroxyborate or peroxycarbonate), phthalic acid and its salts, monoperthalic acid and its salts and esters, ascorbyl stearate, oleoyl sarcosine, alkyl sulfate, dioctyl sulfosuccinate, salicylanilide, domiphen bromide, delmopinol, octapinol and other piperidino derivatives, nisin preparations, chlorite salts; parabens such as methylparaben or propylparaben and mixtures of any of the foregoing. One or more additional antibacterial or preservative agents may optionally be present in the composition in a total amount of from about 0.01 wt. % to about 0.5 wt. %, optionally about 0.05 wt. % to about 0.1 wt. % or about 0.3%.by total weight of the composition.

In some embodiments, the oral care compositions may comprise at least one bicarbonate salt useful for example to impart a “clean feel” to teeth and gums due to effervescence and release of carbon dioxide. Any orally acceptable bicarbonate can be used, including without limitation, alkali metal bicarbonates such as sodium and potassium bicarbonates, ammonium bicarbonate and the like. The one or more additional bicarbonate salts are optionally present in a total amount of about 0.1 wt. % to about 50 wt. %, for example about 1 wt. % to 20 wt. %, by total weight of the composition.

In some embodiments, the oral care compositions also comprise at least one flavorant, useful for example to enhance taste of the composition. Any orally acceptable natural or synthetic flavorant can be used, including without limitation essential oils and various flavoring aldehydes, esters, alcohols, and similar materials, tea flavors, vanillin, sage, marjoram, parsley oil, spearmint oil, cinnamon oil, oil of wintergreen, peppermint oil, clove oil, bay oil, anise oil, eucalyptus oil, citrus oils, fruit oils, sassafras and essences including those derived from lemon, orange, lime, grapefruit, apricot, banana, grape, apple, strawberry, cherry, pineapple, etc., bean- and nut-derived flavors such as coffee, cocoa, cola, peanut, almond, etc., adsorbed and encapsulated flavorants and the like. Also encompassed within flavorants herein are ingredients that provide fragrance and/or other sensory effect in the mouth, including cooling or wanning effects. Such ingredients illustratively include menthol, carvone, menthyl acetate, menthyl lactate, camphor, eucalyptus oil, eucalyptol, anethole, eugenol, cassia, oxanone, a-irisone, propenyl guaiethoi, thymol, linalool, benzaldehyde, cinnamaldehyde, N-ethyl-p-menthan-3-carboxamine, N,2,3-trimethyl-2-isopropylbutanamide, 3-(1-menthoxy)-propane-1,2-diol, cinnamaldehyde glycerol acetal (CGA), menthone glycerol acetal (MGA) and the like. One or more flavorants are optionally present in a total amount of from about 0.01 wt. % to about 5 wt. %, for example, from about 0.03 wt. % to about 2.5 wt. %, optionally about 0.05 wt. % to about 1.5 wt. %, further optionally about 0.1 wt. % to about 0.3 wt. % and in some embodiments in various embodiments from about 0.01 wt. % to about 1 wt. %, from about 0.05 to about 2%, from about 0.1% to about 2.5%, and from about 0.1 to about 0.5% by total weight of the composition.

In some embodiments, the oral care compositions comprise at least one sweetener, useful for example to enhance taste of the composition. Sweetening agents among those useful herein include dextrose, polydextrose, sucrose, maltose, dextrin, dried invert sugar, mannose, xylose, ribose, fructose, levulose, galactose, corn syrup, partially hydrolyzed starch, hydrogenated starch hydrolysate, ethanol, sorbitol, mannitol, xylitol, maltitol, isomalt, aspartame, neotame, saccharin and salts thereof (e.g. sodium saccharin), sucralose, dipeptide-based intense sweeteners, cyclamates, dihydrochalcones, glycerine, propylene glycol, polyethylene glycols, Poloxomer polymers such as POLOXOMER 407, PLURONIC F108, (both available from BASF Corporation), alkyl polyglycoside (APG), polysorbate, PEG40, castor oil, menthol, and mixtures thereof. One or more sweeteners are optionally present in a total amount depending strongly on the particular sweetener(s) selected, but typically 0.005 wt. % to 5 wt. %, by total weight of the composition, optionally 0.005 wt. % to 0.2 wt. %, further optionally 0.05 wt. % to 0.1 wt. % by total weight of the composition.

In some embodiments, the oral care compositions further comprise an agent that interferes with or prevents bacterial attachment, e.g., ethyl lauroyl arginiate (ELA), solbrol or chitosan, as well as plaque dispersing agents such as enzymes (papain, glucoamylase, etc.).

In some embodiments, the oral care compositions also comprise at least one surfactant. Any orally acceptable surfactant, most of which are anionic, cationic, zwitterionic, nonionic or amphoteric, and mixtures thereof, can be used. Examples of suitable surfactants include water-soluble salts of higher fatty acid monoglyceride monosulfates, such as the sodium salt of monosulfated monoglyceride of hydrogenated coconut oil fatty acids; higher alkyl sulfates such as sodium lauryl sulfate, sodium coconut monoglyceride sulfonate, sodium lauryl sarcosinate, sodium lauryl isoethionate, sodium laureth carboxylate and sodium dodecyl benzenesulfonate; alkyl aryl sulfonates such as sodium dodecyl benzene sulfonate; higher alkyl sulfoacetates, such as sodium lauryl sulfoacetate; higher fatty acid esters of 1,2-dihydroxypropane sulfonate; and the substantially saturated higher aliphatic acyl amides of lower aliphatic amino carboxylic compounds, such as those having 12-16 carbons in the fatty acid, alkyl or acyl radicals; and the like. Examples of amides include N-lauryl sarcosine, and the sodium, potassium and ethanolamine salts of N-lauryl, N-myristoyl, or N-palmitoyl sarcosine. Examples of cationic surfactants include derivatives of aliphatic quaternary ammonium compounds having one long alkyl chain containing 8 to 18 carbon atoms such as lauryl trimethylammonium chloride, cetyl pyridinium chloride, cetyl trimethyl ammonium bromide, di-isobutylphenoxyethyldimethylbenzylammonium chloride, coconut alkyltrimethylammonium nitrite, cetyl pyridinium fluoride, and mixtures thereof. Suitable nonionic surfactants include without limitation, poloxamers, polyoxyethylene sorbitan esters, fatty alcohol ethoxylates, alkylphenol ethoxylates, tertiary amine oxides, tertiary phosphine oxides, di alkyl sulfoxides and the like. Others include, for example, non-anionic polyoxyethylene surfactants, such as Polyoxamer 407, Steareth 30, Polysorbate 20, and castor oil; and amphoteric surfactants such as derivatives of aliphatic secondary and tertiary amines having an anionic group such as carboxylate, sulfate, sulfonate, phosphate or phosphonate such as cocamidopropyl betaine (tegobaine), and cocamidopropyl betaine lauryl glucoside; condensation products of ethylene oxide with various hydrogen containing compounds that are reactive therewith and have long hydrocarbon chains (e.g., aliphatic chains of from 12 to 20 carbon atoms), which condensation products (ethoxamers) contain hydrophilic polyoxyethylene moieties, such as condensation products of poly (ethylene oxide) with fatty acids, fatty, alcohols, fatty amides and other fatty moieties, and with propylene oxide and polypropylene oxides. In some embodiments, the oral composition includes a surfactant system that is sodium laurel sulfate (SLS) and cocamidopropyl betaine. One or more surfactants are optionally present in a total amount of about 0.01 wt. % to about 10 wt. %, for example, from about 0.05 wt. % to about 5 wt. %, or from about 0.1 wt. % to about 2 wt. %, e.g 1.5% wt. by total weight of the composition. In some embodiments, the oral composition include an anionic surfactant, e.g., a surfactant selected from sodium lauryl sulfate, sodium ether lauryl sulfate, and mixtures thereof, e.g. in an amount of from about 0.3% to about 4.5% by weight, e.g. 1-2% sodium lauryl sulfate (SLS); and/or a zwitterionic surfactant, for example a betaine surfactant, for example cocamidopropylbetaine, e.g. in an amount of from about 0.1% to about 4.5% by weight, e.g. 0.5-2% cocamidopropylbetaine. Some embodiments comprise a nonionic surfactant in an amount of from 0.5-5%, e.g, 1-2%, selected from poloxamers (e.g., poloxamer 407), polysorbates (e.g., polysorbate 20), polyoxyl hydrogenated castor oil (e.g., polyoxyl 40 hydrogenated castor oil), and mixtures thereof. In some embodiments, the poloxamer nonionic surfactant has a polyoxypropylene molecular mass of from 3000 to 5000 g/mol and a polyoxyethylene content of from 60 to 80 mol %, e.g., the poloxamer nonionic surfactant comprises poloxamer 407. Any of the preceding compositions may further comprise sorbitol, wherein the sorbitol is in a total amount of 10-40% (e.g., about 23%).

In some embodiments, the oral care compositions comprise at least, one foam modulator, useful for example to increase amount, thickness or stability of foam generated by the composition upon agitation. Any orally acceptable foam modulator can be used, including without limitation, polyethylene glycols (PEGs), also known as polyoxyethylenes. High molecular weight PEGs are suitable, including those having an average molecular weight of 200,000 to 7,000,000, for example 500,000 to 5,000,000, or 1,000,000 to 2,500,000, One or more PEGs are optionally present in a total amount of about 0.1 wt. % to about 10 wt. %, for example from about 0.2 wt. % to about 5 wt. %, or from about 0.25 wt. % to about 2 wt. %, by total weight of the composition

In some embodiments, the oral care compositions comprise at least one pH modifying agent. Such agents include acidifying agents to lower pH, basifying agents to raise pH, and buffering agents to control pH within a desired range. For example, one or more compounds selected from acidifying, basifying and buffering agents can be included to provide a pH of 2 to 10, or in various illustrative embodiments, 2 to 8, 3 to 9, 4 to 8, 5 to 7, 6 to 10, 7 to 9, etc. Any orally acceptable pH modifying agent can be used, including without limitation, carboxylic, phosphoric and sulfonic acids, acid salts (e.g., monosodium citrate, disodium citrate, monosodium malate, etc.), alkali metal hydroxides such as sodium hydroxide, carbonates such as sodium carbonate, bicarbonates such as sodium bicarbonate, sesquicarbonates, borates, silicates, bisulfates, phosphates (e.g., monosodium phosphate, trisodium phosphate, monopotassium phosphate, dipotassium phosphate, tribasic sodium phosphate, sodium tripolyphosphate, phosphoric acid), imidazole, sodium phosphate buffer (e.g., sodium phosphate monobasic and disodium phosphate) citrates (e.g. citric acid, trisodium citrate dehydrate), pyrophosphates (sodium and potassium salts) and the like and combinations thereof. One or more pH modifying agents are optionally present in a total amount effective to maintain the composition in an orally acceptable pH range. Compositions may have a pH that is either acidic or basic, e.g., from pH 4 to pH 5.5 or from pH 8 to pH 10. In some embodiments, the amount of buffering agent is sufficient to provide a pH of about 5 to about 9, preferable about 6 to about 8, and more preferable about 7, when the composition is dissolved in water, a mouthrinse base, or a toothpaste base. Typical amounts of buffering agent are about 5% to about 35%, in one embodiment about 10% to about 30%), in another embodiment about 15% to about 25%, by weight of the total composition.

In some embodiments, the oral care compositions also comprise at least one humectant. Any orally acceptable humectant can be used, including without limitation, polyhydric alcohols such as glycerin, sorbitol (optionally as a 70 wt. % solution in water), propylene glycol, xylitol or low molecular weight polyethylene glycols (PEGs) and mixtures thereof. Most humectants also function as sweeteners. In some embodiments, compositions comprise 15% to 70% or 30% to 65% by weight humectant. Suitable humectants include edible polyhydric alcohols such as glycerine, sorbitol, xylitol, propylene glycol as well as other polyols and mixtures of these humectants. Mixtures of glycerine and sorbitol may be used in certain embodiments as the humectant component of the compositions herein. One or more humectants are optionally present in a total amount of from about 1 wt. % to about 70 wt. %, for example, from about 1 wt. % to about 50 wt. %, from about 2 wt. % to about 25 wt. %, or from about 5 wt. % to about 15 wt. %, by total weight of the composition. In some embodiments, humectants, such as glycerin are present in an amount that is at least 20%>, e.g., 20-40%, e.g., 25-35%.

Mouth-feel agents include materials imparting a desirable texture or other feeling during use of the composition. In some embodiments, the oral care compositions comprise at least one thickening agent, useful for example to impart a desired consistency and/or mouth feel to the composition. Any orally acceptable thickening agent can be used, including without limitation, carbomers, also known as carboxyvinyl polymers, carrageenans, also known as Irish moss and more particularly i-carrageenan (iota-carrageenan), cellulosic polymers such as hydroxyethyl cellulose, and water-soluble salts of cellulose ethers (e.g., sodium carboxymethyl cellulose and sodium carboxymethyl hydroxyethyl cellulose), carboxymethylcellulose (CMC) and salts thereof, e.g., CMC sodium, natural gums such as karaya, xanthan, gum arabic and tragacanthin, colloidal magnesium aluminum silicate, colloidal silica, starch, polyvinyl pyrrolidone, hydroxyethyl propyl cellulose, hydroxybutyl methyl cellulose, hydroxypropyl methyl cellulose, and hydroxyethyl cellulose and amorphous silicas, and the like. A preferred class of thickening or gelling agents includes a class of homopolymers of acrylic acid crosslinked with an alkyl ether of pentaerythritol or an alkyl ether of sucrose, or carbomers. Carbomers are commercially available from B. F. Goodrich as the Carbopol© series. Particularly preferred Carbopols include Carbopol 934, 940, 941, 956, 974P, and mixtures thereof. Silica thickeners such as DT 267 (from PPG Industries) may also be used. One or more thickening agents are optionally present in a total amount of from about 0.01 wt. % to 15 wt. %, for example from about 0.1 wt. % to about 10 wt. %, or from about 0.2 wt. % to about 5 wt. %, by total weight of the composition. Some embodiments comprise sodium carboxymethyl cellulose (e.g., from 0.5 wt. %-1.5 wt. %). In certain embodiments, thickening agents in an amount of about 0.5% to about 5.0% by weight of the total composition are used. Thickeners may be present in an amount of from 1 wt % to 15 wt %, from 3 wt % to 10 wt %, 4 wt % to 9 wt %, from 5 wt % to 8 wt %, for example 5 wt %, 6 wt %, 7 wt %, or 8 wt %.

In some embodiments, the oral care compositions comprise at least one colorant. Colorants herein include pigments, dyes, lakes and agents imparting a particular luster or reflectivity such as pearling agents. In various embodiments, colorants are operable to provide a white or light-colored coating on a dental surface, to act as an indicator of locations on a dental surface that have been effectively contacted by the composition, and/or to modify appearance, in particular color and/or opacity, of the composition to enhance attractiveness to the consumer. Any orally acceptable colorant can be used, including FD&C dyes and pigments, talc, mica, magnesium carbonate, calcium carbonate, magnesium silicate, magnesium aluminum silicate, silica, titanium dioxide, zinc oxide, red, yellow, brown and black iron oxides, ferric ammonium ferrocyanide, manganese violet, ultramarine, titaniated mica, bismuth oxychloride, and mixtures thereof. One or more colorants are optionally present in a total amount of about 0.001% to about 20%, for example about 0.01% to about 10% or about 0.1% to about 5% by total weight of the composition.

In some embodiments, the oral care composition further comprises an anti-calculus (tartar control) agent. Suitable anti-calculus agents include, but are not limited to: phosphates and polyphosphates, polyaminopropane sulfonic acid (AM PS), polyolefin sulfonates, polyolefin phosphates, diphosphonates such as azacycloalkane-2,2-diphosphonates (e.g., azacycloheptane-2,2-diphosphonic acid), N-methyl azacyclopentane-2,3-diphosphonic acid, ethane-1-hydroxy-1,1-diphosphonic acid (EHDP) and ethane-1-amino-1,1-diphosphonate, phosphonoalkane carboxylic acids and. Useful inorganic phosphate and polyphosphate salts include monobasic, dibasic and tribasic sodium phosphates. Soluble pyrophosphates are useful anticalculus agents. The pyrophosphate salts can be any of the alkali metal pyrophosphate salts. In certain embodiments, salts include tetra alkali metal pyrophosphate, dialkali metal diacid pyrophosphate, trialkali metal monoacid pyrophosphate and mixtures thereof, wherein the alkali metals are sodium or potassium. The pyrophosphates also contribute to preservation of the compositions by lowering water activity, tetrasodium pyrophosphate (TSPP), tetrapotassium pyrophosphate, sodium tripolyphosphate, tetrapolyphosphate, sodium trimetaphosphate, sodium hexametaphosphate and mixtures thereof. The salts are useful in both their hydrated and unhydrated forms. An effective amount of pyrophosphate salt useful in the present composition is generally enough to provide least 0.1 wt. % pyrophosphate ions, e.g., 0.1 to 3 wt. %, e.g., 0.1 to 2 wt. %, e.g., 0.1 to 1 wt. %, e.g., 0.2 to 0.5 wt. %.

Other useful tartar control agents include polymers and co-polymers. In some embodiments, the oral care compositions include one or more polymers, such as polyethylene glycols, polyvinyl methyl ether maleic acid copolymers, polysaccharides (e.g., cellulose derivatives, for example carboxymethyl cellulose, or polysaccharide gums, for example xanthan gum or carrageenan gum). Acidic polymers, for example polyacrylate gels, may be provided in the form of their free acids or partially or fully neutralized water-soluble alkali metal (e.g., potassium and sodium) or ammonium salts. Certain embodiments include 1:4 to 4:1 copolymers of maleic anhydride or acid with another polymerizable ethylenically unsaturated monomer, for example, methyl vinyl ether (methoxyethylene), having a molecular weight (M.W.) of about 30,000 to about 1,000,000, polyvinyl methyl ether/maleic anhydride (PVM/MA) copolymers such as GANTREZ® (e.g., GANTREZ® S-97 polymer). In some embodiments, the PVM/MA copolymer comprises a copolymer of methyl vinyl ether/maleic anhydride, wherein the anhydride is hydrolyzed following copolymerization to provide the corresponding acid. In some embodiments, PVM/MA copolymer has an average molecular weight (M.W.) of about 30,000 to about 1,000,000, e.g. about 300,000 to about 800,000, e.g., wherein the anionic polymer is about 1-5%, e.g., about 2%, of the weight of the composition. In some embodiments, the anti-calculus agent is present in the composition in an amount of from 0.2 weight % to 0.8 weight %; 0.3 weight % to 0.7 weight %; 0.4 weight % to 0.6 weight %; or about 0.5 weight %, based on the total weight of the composition. Copolymers are available for example as Gantrez AN 139 (M.W. 500,000), AN 1 19 (M.W. 250,000) and S-97 Pharmaceutical Grade (M.W. 70,000), of GAF Chemicals Corporation. Other operative polymers include those such as the 1:1 copolymers of maleic anhydride with ethyl acrylate, hydroxyethyl methacrylate, N-vinyl-2-pyrollidone, or ethylene, the latter being available for example as Monsanto EMA No. 1 103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone. Suitable generally, are polymerized olefinically or ethyl enically unsaturated carboxylic acids containing an activated carbon-to-carbon olefinic double bond and at least one carboxyl group, that is, an acid containing an olefinic double bond which readily functions in polymerization because of its presence in the monomer molecule either in the alpha-beta position with respect to a carboxyl group or as part of a terminal methylene grouping. Illustrative of such acids are acrylic, methacrylic, ethacrylic, alpha-chloroacrylic, crotonic, beta-acryloxy propionic, sorbic, alpha- chlorsorbic, cinnamic, beta-styrylacrylic, muconic, itaconic, citraconic, mesaconic, glutaconic, aconitic, alpha-phenylacrylic, 2-benzyl acrylic, 2-cyclohexylacrylic, angelic, umbellic, fumaric, maleic acids and anhydrides. Other different olefinic monomers copolymerizable with such carboxylic monomers include vinylacetate, vinyl chloride, dimethyl maleate and the like. Copolymers contain sufficient carboxylic salt groups for water-solubility. A further class of polymeric agents includes a composition containing homopolymers of substituted acrylamides and/or homopolymers of unsaturated sulfonic acids and salts thereof, in particular where polymers are based on unsaturated sulfonic acids selected from acrylamidoalykane sulfonic acids such as 2-acrylamide 2 methylpropane sulfonic acid having a molecular weight of about 1,000 to about 2,000,000. Another useful class of polymeric agents includes polyamino acids, particularly those containing proportions of anionic surface-active amino acids such as aspartic acid, glutamic acid and phosphoserine.

In some embodiments, the oral care compositions comprise a saliva stimulating agent useful, for example, in amelioration of dry mouth. Any orally acceptable saliva stimulating agent can be used, including without limitation food acids such as citric, lactic, malic, succinic, ascorbic, adipic, fumaric and tartaric acids, and mixtures thereof. One or more saliva stimulating agents are optionally present in saliva stimulating effective total amount.

In some embodiments, the oral care compositions comprise a nutrient. Suitable nutrients include vitamins, minerals, amino acids, and mixtures thereof. Vitamins include Vitamins C and D, miamine, riboflavin, calcium pantothenate, niacin, folic acid, nicotinamide, pyridoxine, cyanocobalamin, para-aminobenzoic acid, bioflavonoids, and mixtures thereof. Nutritional supplements include amino acids (such as L-tryptophane, L-lysine, methionine, threonine, levocarnitine and L-carnitine), lipotropics (such as choline, inositol, betaine, and linoleic acid), and mixtures thereof.

In some embodiments, the oral care compositions comprise at least one viscosity modifier, useful for example to help inhibit settling or separation of ingredients or to promote re-dispersibility upon agitation of a liquid composition. Any orally acceptable viscosity modifier can be used, including without limitation, mineral oil, petrolatum, clays and organo-modified clays, silicas and the like. One or more viscosity modifiers are optionally present in a total amount of from about 0.01 wt. % to about 10 wt. %, for example, from about 0.1 wt. % to about 5 wt. %, by total weight of the composition.

In some embodiments, the oral care compositions comprise antisensitivity agents, e.g., potassium salts such as potassium nitrate, potassium bicarbonate, potassium chloride, potassium citrate, and potassium oxalate; capsaicin; eugenol; strontium salts; chloride salts and combinations thereof. Such agents may be added in effective amounts, e.g., from about 1 wt. % to about 20 wt. % by weight based on the total weight of the composition, depending on the agent chosen.

In some embodiments, the oral care compositions comprise an antioxidant. Any orally acceptable antioxidant can be used, including butylated hydroxy anisole (BHA), butylated hydroxytoluene (BHT), vitamin A, carotenoids, co-enzyme Q10, PQQ, Vitamin A, Vitamin C, vitamin E, anethole-dithiothione, flavonoids, polyphenols, ascorbic acid, herbal antioxidants, chlorophyll, melatonin, and mixtures thereof.

In some embodiments, the oral care compositions comprise of one or more alkali phosphate salts, e.g., sodium, potassium or calcium salts, e.g., selected from alkali dibasic phosphate and alkali pyrophosphate salts, e.g., alkali phosphate salts selected from sodium phosphate dibasic, potassium phosphate dibasic, dicalcium phosphate dihydrate, calcium pyrophosphate, tetrasodium pyrophosphate, tetrapotassium pyrophosphate, sodium tripolyphosphate, disodium hydrogenorthophosphate, monosodium phosphate, pentapotassium triphosphate and mixtures of any of two or more of these, e.g., in an amount of 0.01-20%, e.g., 0.1-8%, e.g., e.g., 0.1 to 5%, e.g., 0.3 to 2%, e.g., 0.3 to 1%, e.g about 0.01%, about 0.1%, about 0.5%, about 1%, about 2%, about 5%, about 6%, by weight of the composition. In some embodiments, compositions comprise tetrapotassium pyrophosphate, disodium hydrogenorthophosphate, monosodium phosphate, and pentapotassium triphosphate. In some embodiments, compositions comprise tetrasodium pyrophosphate from 0.1-1.0 wt % (e.g., about 0.5 wt %).

In some embodiments, the oral care compositions comprise a source of calcium and phosphate selected from (i) calcium-glass complexes, e.g., calcium sodium phosphosilicates, and (ii) calcium-protein complexes, e.g., casein phosphopeptide-amorphous calcium phosphate. Any of the preceding compositions further comprising a soluble calcium salt, e.g., selected from calcium sulfate, calcium chloride, calcium nitrate, calcium acetate, calcium lactate, and combinations thereof.

In some embodiments, the oral care compositions comprise an additional ingredient selected from: benzyl alcohol, Methylisothizolinone (“MIT”), Sodium bicarbonate, sodium methyl cocoyl taurate (tauranol), lauryl alcohol, and polyphosphate. Some embodiments comprise benzyl alcohol that is present from 0.1-0.8 wt %., or 0.2 to 0.7 wt %, or from 0.3 to 0.6 wt %, or from 0.4 to 0.5 wt %, e.g. about 0.1 wt. %, about 0.2 wt. %, about 0.3 wt %, about 0.4 wt %, about 0.5 wt %, about 0.6 wt %, about 0.7 wt % or about 0.8 wt %.

In some embodiments, the oral care compositions comprise from 5%-40%, e.g., 10%-35%, e.g., about 15%, 25%, 30%, and 35% or more of water.

### EXAMPLES

### Example 1

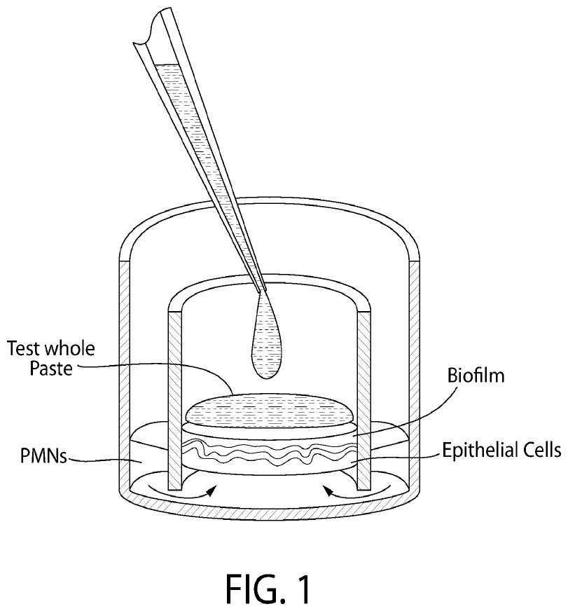

FIG. 1 contains an illustration of the Gingival Crevice Model (GCM). The GCM is useful to assess product health benefits in a cell culture model that closely mimics a gingival crevice. The gingival crevice is home to hundreds of bacterial species along with gingival epithelial cells and neutrophils. Proteomics of secreted or expressed proteins, bacterial impact and odor can be evaluated and used to compare the impact of various compounds and compositions. The GCM combines three components, neutrophil-like cells, biofilm that includes oral bacteria, and oral epithelial tissue.

Neutrophil-like cells: HL60 cells (ATCC #CCLO-240) can be induced to differentiate into a neutrophil-like cell types by contacting the HL60 cells with retinoic acid. HL60 cells are maintained at a cell density of 1×10⁵ cells/mL (Media for HL60 IMEM ATCC #30-2005). Retinoic acid for differentiation of HL60s into neutrophil-like is prepared by dissolving retinoic acid into ETOH to produce a 1 mM solution of retinoic acid in ethanol. When the HL60 cells are to be induced to differentiate into a neutrophil-like cell types by retinoic acid at a concentration of 1 μM (1:1000 dilution of the 1 mM retinoic acid solution), the HL60 cells are brought up to a cell density of 2×10⁵ cells/mL. Differentiation takes 6 days. Differentiated cells, which make up about 60-80% of cells and are referred to in FIG. 1 as PMNs.

Biofilm: Biofilms are created using saliva cultivated on substrates such as HAP discs, poly-D-lysine, or collagen-coated substrates, or in vivo using enamel in an individually made retainer, collagen matrices, and polydimethylsiloxane (PDMS), agarose, agar, poly(ethylene glycol) dimethacrylate (PEGDMA) and 2-methacryloyloxyethyl phosphorylcholine polymer (PMPC) hydrogels. The cultivation of biofilm typically takes 2 days. McBain media supplemented with 5 μg/ml hemin (final concentration) and 1 μg/ml (final concentration) is inoculated with ˜2 mL of human saliva. Salivary biofilms are cultured for ˜16 hours on substrates, for example HAP disks, under suitable growing conditions such as 37° C. under 5% CO².

Oral Epithelial Tissue: There are two types of oral tissue available from MatTek: EpiGingival™ gingival epithelium and EpiOral™ oral (buccal) epithelium. MatTek' s EpiOral and EpiGingival tissues consist of normal, human-derived oral epithelial cells. The cells have been cultured to form multilayered, highly differentiated models of the human buccal (EpiOral) and gingival (EpiGingival) phenotypes. The tissues are cultured on specially prepared cell culture inserts using serum free medium. The EpiOral and EpiGingival tissue models exhibit in vivo-like morphological and growth characteristics which are uniform and highly reproducible. For traditional GCM, the gingival epithelium is preferred. If a cheek model is the goal, the Oral epithelium is used.

Prior to assembly in the GCM, the HL60 cells must be induced with the retinoic acid to differentiate into the neutrophil-like phenotype (PMNs) and the biofilms must be prepared. The preparation of PMNs and biofilms are coordinated so that the PMNs and biofilms are ready following receipt from the supplier and overnight incubation of the MatTek tissue. Upon delivery of the MatTek tissue (epithelial cells) is placed in fresh media in 6 well plates and left to recover overnight in incubator.

On testing day, the preparation of each of the components of the GCM is coordinated so the each of the components of the GCM is ready for testing at the same time.

When testing toothpaste (TP), the product is prepared as a slurry. The TP product is diluted with ultrapure H₂O immediately prior to testing at 1:2 dilution. Mouthwash can be used at full strength.

MatTek media and (FBS) serum are warmed. Tissue and biofilm are treated separately and the GCM is assembled.

Biofilms are treated once with the 1:2 (TP:water) toothpaste slurry for 2 minutes at room temperature while shaking at ˜100 rpm. Following treatment, the biofilms are washed twice in sterile deionized water at 5 minute intervals and then transferred into fresh sterile water to allow the bacteria to recover at 37° C. for ˜3 hours prior to assembly of the GCM and co-incubation with treated cultured epithelial cells.

To treat the MatTek epithelial tissue, the MatTek tissue is removed from the incubator, and each tissue is taken out for treatment with the 1:2 (TP:water) toothpaste slurry in a 24 well plate. Prior to treatment the media is removed for use a baseline control. Each tissue sample is treated with toothpaste dilution for 2 minutes.

Differentiated HL60s (2.5×10\hat{ }5 cells/mL) are prepared for the GCM by centrifuging 300 RPM for 5 minutes in fresh tubes and re-suspending in MatTek media to model a non-inflammatory condition or MatTek+5% FBS to model an inflammatory condition.

Biofilm and epithelial tissue, which have each been treated with the same type of tooth paste dilution, and PMNs are assembles as shown in FIG. 1 and placed in a bacteria-friendly incubator overnight.

After 24 hours, media from experiment is harvested and HL60s/PMNs are spun out (300 RPM, 5 min) and frozen/store at −20 C. Cytokine/chemokines are detected and quantified using Milliplex MagPix kits. Bacterial analysis can be performed on biofilms on HAP discs or other substrates. Alternatively, the biofilms can be stored in −80 C for later analysis.

PMNs can be recovered for analysis. After removing supernatant from cells, the cells are washed two times in cold PBS (300 RPM, 5 min). The PMNs are brought up in 200 uL of fixation buffer (room temp for 10 min or overnight at 4 C) and stained with desired antibody staining procedure.

MatTek tissue can be evaluated after treatment. MTT assay should be done if there is question about cellular toxicity. The tissue is fixed for histological analysis if the location of protein expression is to be assessed. Tissue may be sonicated and analyzed for cytokine analysis if the protein of interest is not secreted.

The GCM was used to evaluate toothpaste compositions including Composition 1 (Comp1), Composition 2 (Comp2), and Composition 3 (Comp3). Comp1 comprises zinc oxide, zinc citrate and stannous fluoride. Zinc oxide was present in the composition at about 1%. Zinc citrate was present in the composition at about 0.5%. Stannous fluoride was present in the composition at about 0.5%. Comp2 comprises zinc oxide, zinc citrate, stannous fluoride and arginine. Zinc oxide was present in the composition at about 1%. Zinc citrate was present in the composition at about 0.5%. Stannous fluoride was present in the composition at about 0.5%. Arginine was present in the composition at about 1.5%. Comp3 comprises zinc oxide, zinc citrate, stannous fluoride and zingerone. Zinc oxide was present in the composition at about 1%. Zinc citrate was present in the composition at about 0.5%. Stannous fluoride was present in the composition at about 0.5%. Zingerone was present in the composition at about 0.1-0.3%.

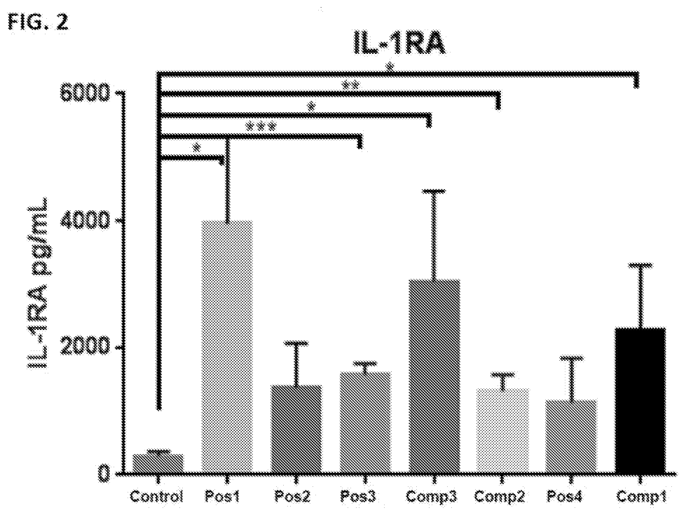

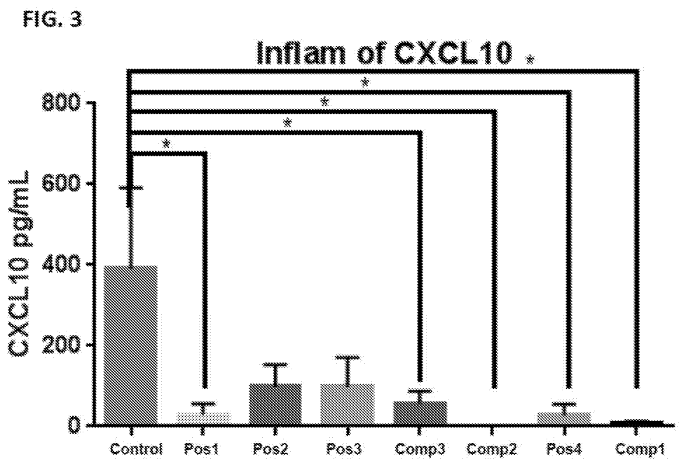

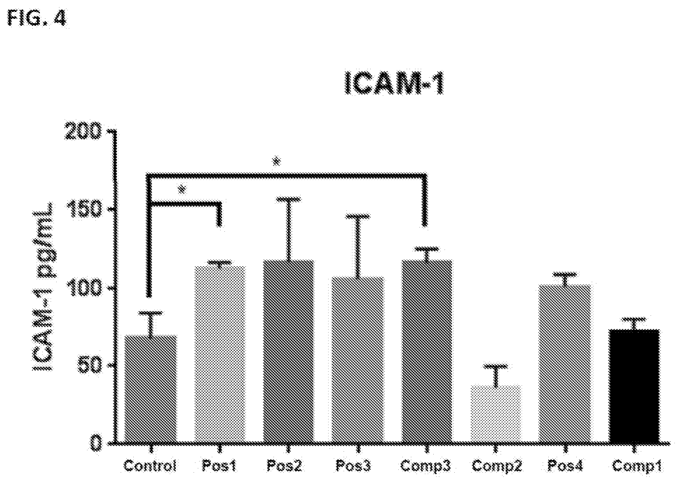

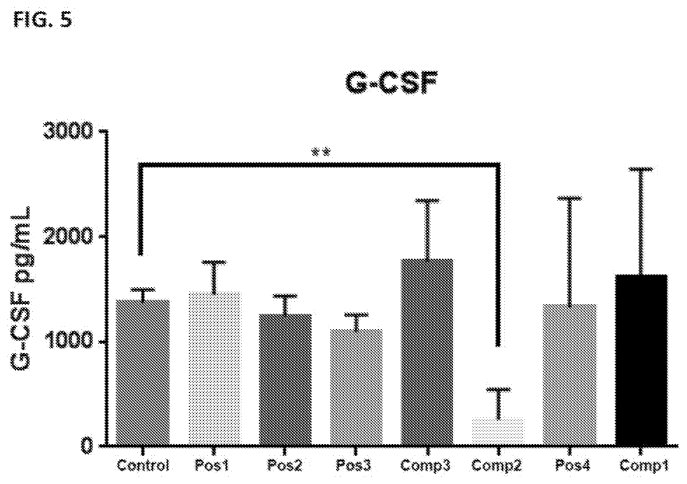

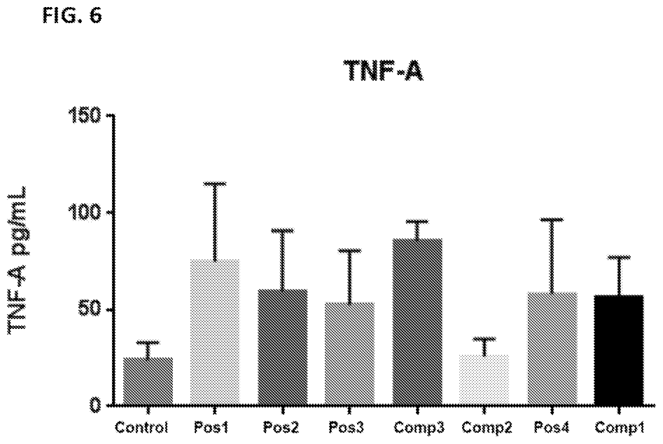

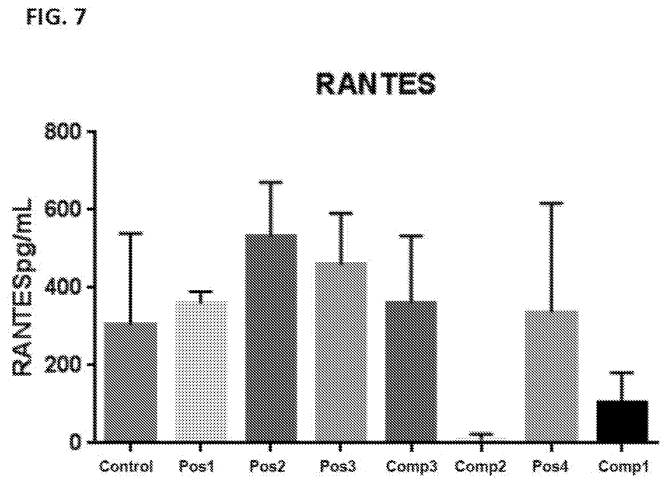

Data from the GCM experiment is shown in FIGS. 2-7. In each figure, control corresponds to the negative control. Pos1-Pos4 refer to control formulations. Comp1, Comp2 and Comp3 correspond to the tested toothpastes. IL-1RA (FIG. 2), CXCL10 (FIG. 3), ICAM-1 (FIG. 4), G-CSF (FIG. 5), TNFa (FIG. 6) and RANTES (FIG. 7) levels in response to the treatment with the various formulations were measured.

As shown in FIGS. 2 and 3, results of treatment in the GCM with Comp1 showed an induction of IL-1Ra (FIG. 2), which is a protein that reduces signals associated with neutrophil recruitment, and a reduction of CXCL10 (FIG. 3), which is a protein that promotes neutrophil recruitment. As shown in FIGS. 2, 3 and 5, results of treatment in the GCM with Comp2 showed an induction of IL-1Ra (FIG. 2), which is a protein that reduces signals associated with neutrophil recruitment, and a reduction of CXCL10 (FIG. 3) and G-CSF (FIG. 5), which are proteins that promote neutrophil recruitment. As shown in FIGS. 2, 3 and 4, results of treatment in the GCM with Comp3 showed an induction of IL-1Ra (FIG. 2) and sICAM (FIG. 4), which are proteins that reduce signals associated with neutrophil recruitment, and a reduction in levels of CXCL10 (FIG. 3), which is a protein that promotes neutrophil recruitment.

Comp2 reduced G-CSF (FIG. 5), which is one of the main neutrophil chemokines and is involved in a key inflammatory pathway that has been shown to be important in the progression of periodontal disease. Comp2 showed a trend in reduction of additional neutrophil chemokines/inflammatory cytokines including TNF-A (FIG. 6) and RANTES (FIG. 7).

### Example 2

Oral compositions that comprise arginine are disclosed in WO/2011/123123, which corresponds to U.S. Pat. No. 8,652,495, which are both incorporated herein by reference. In some embodiments, the oral care composition comprises an orally acceptable vehicle; zinc oxide particles; and arginine. In some such embodiments, zinc oxide particles have a median particle size of from 1 to 7 microns; in some such embodiments, the zinc oxide particles have a particle size distribution of 3 to 4 microns, a particle size distribution of 5 to 7 microns, a particle size distribution of 3 to 5 microns, a particle size distribution of 2 to 5 microns, or a particle size distribution of 2 to 4 microns. In some of these variously described embodiments, the zinc oxide particles are present in an amount of up to 5% by weight, such as from 0.5 to 2% by weight based on the total weight of the oral care composition. In some of these variously described embodiments, the zinc oxide particles the source of zinc oxide particles is selected from a powder, a nanoparticle solution; a nanoparticle suspension; a capsule; and a bead. In some of these variously described embodiments, the oral care composition further comprises at least one additional metal oxide selected from stannous oxide, titanium oxide, calcium oxide, copper oxide and iron oxide or a mixture thereof. In some of these variously described embodiments, the arginine is L-arginine. In some of these variously described embodiments, the arginine is present in an amount of up to 5% by weight, such as from 0.5 to 5% by weight, such 2.5 to 4.5% by weight as based on the total weight of the oral care composition. In some of the various embodiments, the oral composition further comprises in addition to arginine, at least one or more amino acids is selected from cysteine, leucine, isoleucine, lysine, L-lysine, alanine, asparagine, aspartate, phenylalanine, glutamate, glutamic acid, threonine, glutamine, tryptophan, glycine, valine, proline, serine, tyrosine, and histidine, and a combination of two or more thereof. In some of these variously described embodiments, the oral care composition further comprises a polymeric adherent material. In some of these variously described embodiments, the source of zinc oxide particles is a capsule, and the capsule comprises a polymeric adherent material and in some such embodiments, the polymeric adherent material comprises one or more cellulose polymers, such as embodiments in which at least one of said one or more cellulose polymers is a hydroxyalkyl cellulose polymer selected from hydroxypropylmethyl cellulose (HPMC), hydroxyethylpropyl cellulose (HEPC), hydroxybutylmethyl cellulose (HBMC), and carboxymethyl cellulose (CMC). In some of these variously described embodiments, the polymeric adherent material comprises a mixture of two hydroxyalkyl cellulose polymers having different molecular weights and the zinc oxide which is encapsulated in the mixture of two hydroxyalkyl cellulose polymers. In some of these variously described embodiments, the polymeric adherent material comprises one or more polymers selected from a poly (ethylene oxide) polymer, polyvinylpyrrolidone (PVP), polyethylene glycol (PEG)/polypropylene glycol (PPG) copolymer, ethylene oxide (EO)—propylene oxide (PO) block copolymers, ester gum, shellac, pressure sensitive silicone adhesives, methacrylates, or mixtures thereof. In some of these variously described embodiments, the oral care composition is a dentifrice composition, such as for example, a toothpaste or a gel. In some of these variously described embodiments, the oral care composition is formulated into a form selected from a mouth rinse, a gum, a dissolvable lozenge, and a dissolvable film.

### Example 3

Oral compositions that comprise arginine are disclosed in WO 2014/088572, which corresponds to US 2015/0313813, which are both incorporated herein by reference. In some embodiments the oral care composition comprises: from about 0.05 to about 5% by weight, of a zinc ion source; a fluoride ion source in an amount effective to deliver from about 500 to about 5,000 ppm fluoride, and from about 0.1 to about 10%, by weight, of arginine. In some such embodiments, the zinc ion source is selected from zinc citrate, zinc sulfate, zinc silicate, zinc lactate, zinc phosphate, zinc oxide, and combinations thereof, for example, in an amount effective to inhibit erosion. In some such embodiments, the oral composition is in the form of a dentifrice comprising an abrasive. In some such embodiments, the amount of zinc is 0.5 to 4% by weight. In some such embodiments, the compositions may further comprise one or more abrasives, one or more humectants, and one or more surfactants. In some such embodiments, the compositions may further comprise an effective amount of one or more alkali phosphate salts and/or an effective amount of one or more antibacterial agents and/or a whitening agent. In some such embodiments, the composition comprises zinc phosphate and one or more other sources of zinc ion. In some such embodiments, the pH of the composition is basic. In some such embodiments, the composition may comprise, in a silica abrasive dentifrice base: 1 to 3% zinc citrate; 1 to 8% arginine; 700 to 2000 ppm fluoride; and 2 to 8% alkali phosphate salts selected from sodium phosphate dibasic, potassium phosphate dibasic, dicalcium phosphate dihydrate, tetrasodium pyrophosphate, tetrapotassium pyrophosphate, calcium pyrophosphate, sodium tripolyphosphate, and a combination of two or more thereof.

### Example 4

Oral compositions that comprise arginine are disclosed in WO 2015/094849, which corresponds to US 2016/0338921, which are both incorporated herein by reference. In some embodiments the oral care composition comprises: arginine, in free or salt form; and zinc oxide and zinc citrate. In some embodiments, the arginine is present in an amount of 0.5 weight % to 3 weight %, such as 1 weight % to 2.85 weight %, such as 1.17 weight % to 2.25 weight %, such as 1.4 weight % to 1.6 weight %, such as about 1.5 weight %, based on the total weight of the composition. In some embodiments set out above, the total concentration of zinc salts in the composition is 0.2 weight % to 5 weight %, based on the total weight of the composition. In some embodiments set out above, the molar ratio of arginine to total zinc salts is 0.05:1 to 10:1. In some embodiments set out above, the composition comprises zinc oxide in an amount of 0.5 weight % to 1.5 weight %, such as 1 weight %, and zinc citrate in an amount of 0.25 weight % to 0.75 weight %, such as 0.5 weight %, based on the total weight of the composition. In some embodiments set out above, the weight ratio of zinc oxide to zinc citrate is 1.5:1 to 4.5:1, optionally 1.5:1 to 4:1, 1.7:1 to 2.3:1, 1.9:1 to 2.1:1, or about 2:1.

### Example 5

Oral compositions that comprise arginine are disclosed in WO 2017/003844, which corresponds to US 2018/0021234, which are both incorporated herein by reference. In some embodiments, the oral care composition comprises: arginine, zinc oxide and zinc citrate and a fluoride source. In some embodiments, the arginine has the L-configuration. In some embodiments, the arginine is present in an amount corresponding to 0.1% to 15%, or 0.1% to 8%, or about 5.0 wt. %, or about 8.0 wt. %, or about 1.5 wt. %, based on the total weight of the composition, the weight of the arginine acid being calculated as free form. In some embodiments, the arginine is in free form or partially or wholly salt form. In some embodiments set out above, the ratio of the amount of zinc oxide (by wt %) to zinc citrate (by wt %) is 2:1, 2.5:1, 3:1, 3.5:1 or 4:1, wherein the ratio is by wt. of the overall composition. In some embodiments, the zinc citrate is in an amount of from 0.25 to 1.0 wt % and zinc oxide may be present in an amount of from 0.75 to 1.25 wt % or the zinc citrate is in an amount of about 0.5 wt % and zinc oxide is present in an amount of about 1.0%, based on the total weight of the composition. In some embodiments set out above, the fluoride source is sodium fluoride or sodium monofluorophosphate. In some such embodiments, the sodium fluoride or sodium monofluorophosphate is from 0.1 wt. %-2 wt. % based on the total weight of the composition. In some embodiments, the sodium fluoride or sodium monofluorophosphate is a soluble fluoride salt which provides soluble fluoride in amount of 50 to 25,000 ppm fluoride, such as in an amount of about 1000 ppm-1500 ppm, for example in an amount of about 1450 ppm. In some embodiments the fluoride source is sodium fluoride in an amount about 0.32% by wt. based on the total weight of the composition. In some embodiments, the fluoride source is stannous fluoride. Some embodiments set out above further comprise a preservative selected from: benzyl alcohol, Methylisothizolinone (“MIT”), Sodium bicarbonate, sodium methyl cocoyl taurate (tauranol), lauryl alcohol, and polyphosphate. Some embodiments set out above further comprise benzyl alcohol in an amount of from 0.1-0.8% wt %, or from 0.3-0.5% wt %, or about 0.4 wt % based on the total weight of the composition. In some embodiments, the oral care composition comprises about 1.0% zinc oxide, about 0.5% zinc citrate, about 1.5% L-arginine, about 1450 ppm sodium fluoride, and optionally about benzyl alcohol 0.1 wt. % and/or about 5% small particle silica (e.g., AC43), based on the total weight of the composition. In some embodiments, the oral care composition comprises about 1.0% zinc oxide, about 0.5% zinc citrate, about 5% L-arginine, about 1450 ppm sodium fluoride, and optionally about benzyl alcohol 0.1 wt. % and/or about 5% small particle silica (e.g., AC43), based on the total weight of the composition. In some embodiments set out above, the oral care composition may comprise about 1.0% zinc oxide, about 0.5% zinc citrate, about 1.5% L-arginine, about 0.22%-0.32% sodium fluoride, about 0.5% tetrasodium pyrophosphate, and optionally about benzyl alcohol 0.1 wt. %, based on the total weight of the composition. In some embodiments set out above, the oral care composition may be any of the following oral care compositions selected from the group consisting of: a toothpaste or a dentifrice, a mouthwash or a mouth rinse, a topical oral gel, and a denture cleanser.

### Example 6

Oral compositions that comprise arginine are disclosed in WO 2017/003856, which is incorporated herein by reference. In some embodiments, the oral care composition comprises: arginine in free or salt form, zinc oxide and zinc citrate and a fluoride source comprising stannous fluoride. In some embodiments, the arginine has the L-configuration. In some embodiments, the arginine is present in an amount corresponding to 0.1% to 15%, or 0.1% to 8%, or about 5.0 wt. %, or about 8.0 wt. %, or about 1.5 wt. %, based on the total weight of the composition, the weight of the arginine acid being calculated as free form. In some embodiments, the arginine is in free form or partially or wholly in salt form. In some embodiments set out above, the ratio of the amount of zinc oxide (by wt. %) to zinc citrate (by wt. %) is 2:1, 2.5:1, 3:1, 3.5:1 or 4:1, wherein the ratio is by weight of the overall composition. In some embodiments set out above, the zinc citrate is in an amount of from 0.25 to 1.0 wt. % and zinc oxide may be present in an amount of from 0.75 to 1.25 wt. % or the zinc citrate is in an amount of about 0.5 wt. % and zinc oxide is present in an amount of about 1.0 wt. %, based on the total weight of the composition. In some embodiments set out above, the fluoride source further comprises at least one member selected from the group of: sodium fluoride, potassium fluoride, sodium monofluorophosphate, sodium fluorosilicate, ammonium fluorosilicate, amine fluoride (e.g., N′-octadecyltrimethylendiamine-N,N,N′-tris(2-ethanol)-dihydrofluoride), ammonium fluoride, titanium fluoride, hexafluorosulfate, and combinations thereof. In some embodiments set out above, the stannous fluoride is present in an amount from 0.1 wt. % to 2 wt. % based on the total weight of the composition. In some embodiments set out above, the stannous fluoride is a soluble fluoride salt which provides soluble fluoride in amount of 50 to 25,000 ppm fluoride, or about 750-7000 ppm, or about 1000-5500 ppm, or about 5000 ppm. In some embodiments, the oral care composition comprises about 1.0% zinc oxide, about 0.5% zinc citrate, about 1.5% L-arginine, about 750-7000 ppm fluoride; and optionally, about 5% small particle silica (e.g., AC43), based on the total weight of the composition. In some embodiments, the oral care composition comprises about 1.0% zinc oxide, about 0.5% zinc citrate, about 750-7000 ppm stannous fluoride; and optionally about 39.2% glycerin based on the total weight of the composition. In some embodiments set out above, the oral care composition may comprise about 1.0% zinc oxide, about 0.5% zinc citrate, about 1.5% L-arginine, stannous fluoride, and optionally about benzyl alcohol 0.1 wt. %, based on the total weight of the composition. In some embodiments set out above, the oral care composition may be any of the following oral compositions selected from the group consisting of: a toothpaste or a dentifrice, a mouthwash or a mouth rinse, a topical oral gel, and a denture cleanser.

### Example 7

Oral compositions that comprise arginine are disclosed in WO 2017/223169, which is incorporated herein by reference. In some embodiments, the oral care composition comprises: arginine in free or salt form, zinc oxide and zinc citrate and a fluoride source comprising stannous fluoride. In some embodiments, the oral care compositions comprise zingerone, zinc oxide, zinc citrate; and a stannous fluoride. In some embodiments, the zingerone is present in an amount of from 0.01% to 1%, based on the total weight of the composition. In some embodiments, the ratio of the amount of zinc oxide (by wt %) to zinc citrate (by wt %) is 2:1, 2.5:1, 3:1, 3.5:1 or 4:1, based on the total weight of the composition. In some embodiments, the zinc citrate is present in an amount of from 0.25 to 1.0 wt % and zinc oxide is present in an amount of from 0.75 to 1.25 wt %, based on the total weight of the composition. In some embodiments, the zinc citrate is present in an amount of about 0.5 wt % and zinc is present in an amount of about 1.0% based on the total weight of the composition. In some embodiments, the stannous fluoride is present in an amount of 0.1 wt. % to 2 wt. %, based on the total weight of the composition. Some embodiments further comprise synthetic amorphous precipitated abrasive silica in an amount of from 1%-25% by wt. based on the total weight of the composition and/or a high cleaning silica in an amount of from 1 wt %-15 wt %, based on the total weight of the composition. Some embodiments further comprise an effective amount of one or more alkali phosphate salts, for example sodium tripolyphosphate in an amount of from 1-5 wt %, based on the total weight of the composition. Some embodiments further comprise citric acid in an amount of from 0.1-3 wt. %, and citrate ion, for example trisodium citrate dihydrate, in an amount of from 0.1-5 wt. %, based on the total weight of the composition. Some embodiments further comprise carboxymethyl cellulose in an amount of from 0.1 wt. %-1.5 wt. %, based on the total weight of the composition. Some embodiments further comprise an anionic surfactant, e.g., sodium lauryl sulfate, in an amount of from 0.5-5% by weight, based on the total weight of the composition. Some embodiments further comprise an amphoteric surfactant in an amount of from 0.5-5%, based on the total weight of the composition. Some embodiments further comprise a PVM/MA copolymer, such as for example a Gantrez polymer, in an amount of from 0.1-5 wt. %, based on the total weight of the composition. Some embodiments further comprise microcrystalline cellulose/sodium carboxymethylcellulose. Some embodiments further comprise one or both of polyethylene glycol in an amount of from 1-6%; and propylene glycol in an amount of from 1-6%, based on the total weight of the composition. Some embodiments further comprise polyvinylpyrrolidone (PVP) in an amount of from 0.5-3 wt. %, based on the total weight of the composition. Some embodiments further comprise from 5% -40% free water by weight, based on the total weight of the composition. Some embodiments further comprise one or more thickening agents, e.g. sodium carboxymethyl cellulose and sodium carboxymethyl hydroxyethyl cellulose,

In some embodiments, the oral care composition comprises: about 0.1-0.3% zingerone; about 1.0% zinc oxide; about 0.5% zinc citrate, and about 0.4%-0.5% stannous fluoride.

In some embodiments, the oral care composition comprises: about 0.1-0.3% zingerone; about 1.0% zinc oxide; about 0.5% zinc citrate, about 0.4%-0.5% stannous fluoride; and about 1.2% abrasive silica and may, in some such embodiments, further comprise about 7% wt % high cleaning silica, based on the total weight of the composition, and/or a surfactant system comprising one or both of an anionic surfactant in an amount of from 0.5-5%, by weight; and/or an amphoteric surfactant in an amount of from 0.5-5% by weight, based on the total weight of the composition. Some embodiments further comprise sodium tripolyphosphate in an amount of from 1-5 wt %, based on the total weight of the composition and/or sodium phosphate in an amount of from 0.5 wt %-5 wt %, based on the total weight of the composition. Examples of the oral composition include a toothpaste or a dentifrice, a mouthwash or a mouth rinse, a topical oral gel, a chewing gum, or a denture cleanser.

### Example 8

A dentifrice composition having the formula of Table A was prepared. The compositions had varying amounts of zinc oxide (varying from 0 to 2 wt %) and of amino acid (varying from 0 to 5 wt %). One example of a dentifrice composition comprised 1 wt % zinc oxide powder, the ZnO being encapsulated, at a 50 wt % loading, into a polymer film, in particular into a polymer film comprising a combination of two different molecular weight HPMC materials, comprising Methocel E5 and Methocel E50. The combined zinc oxide/polymer film comprised 1 wt % ZnO and 1 wt % polymer film, each weight being based on the total weight of the composition. The exemplified dentifrice composition comprised 4.3 wt % L-arginine.

TABLE A

| Ingredient | % w/w | |
|---|---|---|
| Sorbitol | Q.S. | Q.S. |
| Water | 11.994 | 11.994 |
| Silica - Zeodent 105 | 10.000 | 10.000 |
| Silica - Zeodent 114 | 10.00 | 10.00 |
| Polyethylene glycol 600 | 3.00 | 3.00 |
| Silica - Zeodent 165 | 2.75 | 2.75 |
| ZnO | 0-2 | 1* |
| Sodium lauryl sulfate | 1.500 | 1.500 |
| Cocamidopropyl Betaine | 1.250 | 1.250 |
| Flavor | 1.150 | 1.150 |
| Titanium Dioxide | 0.750 | 0.750 |
| Sodium CMC - Type 7MF | 0.065 | 0.065 |
| Arginine | 0-5 | 4.3 |
| Sodium Saccharin | 0.270 | 0.270 |
| Sodium Fluoride | 0.243 | 0.243 |

*ZnO in polymer form - 50 wt % loading into polymer film comprising Methocel E5 and Methocel E50 (combined ZnO 1%/polymer -1%)

### Example 9

Test dentifrices comprising 1% and 2% zinc citrate in combination with 5% arginine, 1450 ppm fluoride, and phosphates are prepared as shown in Table B:

TABLE B

| Ingredient | Zinc 1% | Zinc 2% |
|---|---|---|
| PEG600 | 3.00 | 3.00 |
| CMC-7 | 0.65 | 0.65 |
| Xanthan | 0.20 | 0.20 |
| Sorbitol | 28.4 | 27.4 |
| Glycerin | 20.0 | 20.0 |
| Saccharin | 0.30 | 0.30 |
| TSPP | 0.50 | 0.50 |
| cop Phosphate | 0.25 | 0.25 |
| dibasic Phosphate | 3.50 | 3.50 |
| Na Fluoride | 0.32 | 0.32 |
| Water | QS | QS |
| TiO2 | 0.50 | 0.50 |
| Abrasive silica | 8.00 | 8.00 |
| Thickener silica | 8.00 | 8.00 |
| L-Arginine | 5.00 | 5.00 |
| SLS | 1.50 | 1.50 |
| Brighter Flavor K91-5661 | 1.20 | 1.20 |
| Zinc Citrate | 1.00 | 2.00 |

### Example 10

Test dentifrices comprising arginine, zinc oxide, zinc citrate and a source of fluoride were prepared as shown in Tables C-G:

TABLE C

| Ingredient | Compound I |
|---|---|
| Humectants | 20.0-25.0 |
| Non-ionic surfactant | 1.0-2.0 |
| Amphoteric surfactant | 3.0-4.0 |
| Flavoring/fragrance/coloring agent | 2.0-3.0 |
| Polymers | 10.0-15.0 |
| pH adjusting agents | 1.5-3.0 |
| Precipitated Calcium Carbonate | 35 |
| Zinc citrate trihydrate | 0.5 |
| Zinc oxide | 1.0 |
| Sodium Fluoride -USP, EP | 0.32 |
| Arginine Bicarbonate | 13.86 |
| Demineralized water | QS |

TABLE D

| Ingredient | Compound A | Compound B | Compound C | Compound D |
|---|---|---|---|---|
| Humectants | 25.0-40.0 | 25.0-40.0 | 25.0-40.0 | 25.0-40.0 |
| Anionic surfactant | 1.0-3.0 | 1.0-3.0 | 1.0-3.0 | 1.0-3.0 |
| Flavoring/fragrance/ | 2.5-4.0 | 2.5-4.0 | 2.5-4.0 | 2.5-4.0 |
| coloring agent | | | | |
| Polymers | 4.0-6.0 | 4.0-6.0 | 4.0-6.0 | 4.0-6.0 |
| pH adjusting agents | 5.0-6.0 | 5.0-6.0 | 5.0-6.0 | 5.0-6.0 |
| Synthetic Amorphous | 16.00 | 21.37 | 17.92 | 7.81 |
| Precipitated Silica | | | | |
| Alumina | 0.02 | 0.01 | 0.01 | 0.01 |
| Silica | — | — | — | 15.0 |
| Lauryl alcohol | 0.02 | 0.02 | 0.02 | 0.02 |
| Zinc citrate | 0.5 | 0.5 | 0.5 | 0.5 |
| Zinc oxide | 1.0 | 1.0 | 1.0 | 1.0 |
| Sodium Fluoride - USP, EP | 0.32 | 0.32 | 0.32 | 0.32 |
| L-Arginine Bicarbonate | 5.0 | 5.0 | 5.0 | 5.0 |
| Demineralized water | QS | QS | QS | QS |

TABLE E

| Ingredient | Compound E | Compound F | Compound G |
|---|---|---|---|
| Humectants | 25.0-40.0 | 25.0-40.0 | 25.0-40.0 |
| Anionic surfactant | 1.0-3.0 | 1.0-3.0 | 1.0-3.0 |
| Non-ionic surfactant | 0.1-1.0 | 0.1-1.0 | 0.1-1.0 |
| Amphoteric surfactant | 0.1-1.0 | 0.1-1.0 | 0.1-1.0 |
| Flavoring/fragrance/ | 4.0-6.0 | 4.0-6.0 | 4.0-6.0 |
| coloring agent | | | |
| Polymers | 0.1-2.0 | 0.1-2.0 | 0.1-2.0 |
| pH adjusting agents | 5.0-6.0 | 5.0-6.0 | 5.0-6.0 |
| Thickener | 6.0 | 6.5 | 7.0 |
| Alumina | 0.1 | 0.1 | 0.1 |
| Synthetic Amorphous | 17.6 | 8.8 | 22.4 |
| Precipitated Silica | | | |
| Silica | — | 15.0 | — |
| Benzyl alcohol | 0.1 | 0.1 | 0.1 |
| Synthetic Amorphous Silica | 5.0 | 5.0 | 5.0 |
| Zinc citrate | 0.5 | 0.5 | 0.5 |
| Zinc oxide | 1.0 | 1.0 | 1.0 |
| Sodium Fluoride - USP, EP | 0.32 | 0.32 | 0.32 |
| L-Arginine Bicarbonate | 1.5 | 1.5 | 1.5 |
| Demineralized water | QS | QS | QS |

TABLE F

| Ingredient | Compound H | Compound I |
|---|---|---|
| Humectants | 45.0-55.0 | 35.0-45.0 |
| Abrasives | 14.0-36.0 | 9.0-11.0 |
| Anionic surfactant | 1.0-3.0 | 1.0-3.0 |
| Non-ionic surfactant | 0.1-1.0 | — |
| Amphoteric surfactant | 1.0-2.0 | — |
| Flavoring/fragrance/ | 1.0-3.0 | 2.0-4.0 |
| coloring agent | | |
| Polymers | 0.1-2.0 | 3.0-8.0 |
| pH adjusting agents | 0.1-2.0 | 4.0-8.0 |
| Silica Thickener | 5.0 | 5.0-10.0 |
| Benzyl alcohol | 0.1 | — |
| Zinc citrate trihydrate | 0.5 | 0.5 |
| Zinc oxide | 1.0 | 1.0 |
| Sodium Fluoride - USP, EP | 0.32 | 0.32 |
| L-Arginine | 1.5 | 5.0 |
| Demineralized water | QS | QS |

TABLE G

| Ingredient | Compound J | Compound K | Compound L |
|---|---|---|---|
| Humectants | 20.0-50.0 | 20.0-50.0 | 20.0-50.0 |
| Abrasives | 5.0-20.0 | 5.0-20.0 | 5.0-20.0 |
| Anionic surfactant | 1.0-3.0 | 1.0-3.0 | 1.0-3.0 |
| Non-ionic surfactant | 0.1-1.0 | 0.1-1.0 | 0.1-1.0 |
| Amphoteric surfactant | 0.1-2.0 | 0.1-2.0 | 0.1-2.0 |
| Flavoring/fragrance/ | 1.0-5.0 | 1.0-5.0 | 1.0-5.0 |
| coloring agent | | | |
| Polymers | 0.1 -2.0 | 0.1-2.0 | 0.1-2.0 |
| pH adjusting agents | 0.1-2.0 | 0.1-2.0 | 0.1-2.0 |
| Thickener | 6.0 | 6.5 | 7.0 |
| Dental type silica | — | — | 15.0 |
| High cleaning silica | — | 15.0 | — |
| Synthetic Abrasives | 10.0 | — | — |
| Synthetic Amorphous Silica | 5.0 | 5.0 | 5.0 |
| Benzyl alcohol | 0.4 | 0.4 | 0.4 |
| Zinc citrate trihydrate | 0.5 | 0.5 | 0.5 |
| Zinc oxide | 1.0 | 1.0 | 1.0 |
| Sodium Fluoride - USP, EP | 0.32 | 0.32 | 0.32 |
| L-Arginine | 1.5 | 1.5 | 1.5 |
| Demineralized water | QS | QS | QS |

### Example 11

Test dentifrices comprising arginine, zinc oxide, zinc citrate and stannous fluoride were prepared as shown in Table H:

TABLE H
Ingredient

| | | | |
|---|---|---|---|
| Humectants | 20.0-60.0 | 20.0-50.0 | 2.0.0-50.0 |
| Abrasives | 10.0-40.0 | 5.0-20.0 | 5.0-20.0 |
| Anionic surfactant | 1.0-3.0 | 1.0-3.0 | 1.0-3.0 |
| Amphoteric surfactant | 0.5-1.5 | 0.1-2.0 | 0.1-2.0 |
| Flavoring/fragrance/ | 0.5-5.0 | 1.0-5.0 | 1.0-5.0 |
| coloring agent | | | |
| Polymers | 1.0-10.0 | 0.1-2.0 | 0.1-2.0 |
| pH adjusting agents | 1.0-10.0 | 0.1-2.0 | 0.1-2.0 |
| Zinc citrate | 0.25-1.0 | 0.5 | 0.5 |
| Zinc oxide | 0.75-1.25 | 1.0 | 1.0 |
| Stannous Fluoride | 0.1-1.0 | 0.32 | 0.32 |
| L-Arginine | 0.1-10.0 | 1.5 | 1.5 |
| Demineralized water | QS | QS | QS |

### Example 12

Test dentifrices comprising arginine, zinc oxide, zinc citrate and stannous fluoride were prepared as shown in Table I:

TABLE I
Ingredient

| | | | |
|---|---|---|---|
| Demineralized water | 8.8 | 8.8 | 8.8 |
| Sodium Saccharin | 0.8 | 0.8 | 0.8 |
| Trisodium citrate dihydrate | 3.0 | 3.0 | 3.0 |
| Citric acid anhydrous | 0.6 | 0.6 | 0.6 |
| Stannous fluoride | 0.454 | 0.454 | 0.454 |
| Zinc oxide | 1.0 | 1.0 | 1.0 |
| 99.0-101.0% glycerin | 40.9 | 40.9 | 40.9 |
| Polyethylene glycol | 3.0 | 3.0 | 3.0 |
| Propylene glycol | 4.0 | 4.0 | 4.0 |
| Thickeners (including xanthan gum, | 1.4 | 1.4 | 1.4 |
| carboxymethyl cellulose, | | | |
| microcrystalline cellulose NaCMV) | | | |
| PVP | 1.25 | 1.25 | 1.25 |
| Dye | 0.002 | 0.002 | 0.002 |
| Abrasives (including synthetic amorphous | 24.0 | 24.0 | 24.0 |
| silica, precipitated silica: | | | |
| high cleaning silica, silicon dioxide) | | | |
| Sodium lauryl sulfate powder | 1.75 | 1.75 | 1.75 |
| Cocamidopropyl betaine (30% solution) | 1.0 | 1.0 | 1.0 |
| Gantrez S-97 (16.5% solution) | 0.606 | 0.606 | 0.606 |
| Titanium dioxide coated mica | 0.15 | 0.15 | 0.15 |
| 85% syrupy phosphoric acid food grade | 0.60 | 0.60 | 0.60 |
| Sodium triphosphate tribasic 12-hydrate | 1.0 | 1.0 | 1.0 |
| Zinc citrate trihydrate | 0.5 | 0.5 | 0.5 |
| Sodium tripolyphosphate | 3.0 | 3.0 | 3.0 |
| FCC grade flavor | 2.2 | 2.1 | 1.9 |
| zingerone | — | 0.10 | 0.30 |

1. A method of increasing proteins that reduce signals associated with neutrophil recruitment and reducing proteins that induce neutrophil recruitment within an individual's gingival crevice, the method comprising applying to the individual's oral cavity in an amount effective to increase proteins that reduce signals associated with neutrophil recruitment and reduce proteins that induce neutrophil recruitment within an individual's gingival crevice, an oral care composition comprising: zinc oxide, zinc citrate, stannous fluoride, and optionally arginine and/or zingerone.

2. The method of claim 1 wherein the oral care composition is a toothpaste.

3. The method of claim 2 wherein:
the zinc oxide is present in an amount of from 0.75 to 1.25 wt % based on the total weight of the composition,
the zinc citrate is present in an amount of from 0.25 to 1.0 wt % based on the total weight of the composition, and
the stannous fluoride is present in an amount of 0.1 wt. % to 2 wt. % based on the total weight of the composition.

4. The method of claim 3 wherein zingerone is present in an amount of from 0.01% to 1%, based on the total weight of the composition.

5. The method of claim 3 wherein arginine is present in an amount of from 0.1% to 15%, based on the total weight of the composition, the weight of the basic amino acid being calculated as free form.

6. The method of claim 5 wherein the arginine is L-arginine.

7. The method of claim 5 wherein the arginine is in free form.

8. The method of claim 5 wherein the arginine is in salt form.

9. The method of claim 1, wherein the ratio of the amount of zinc oxide (by wt %) to zinc citrate (by wt %) is 2:1, 2.5:1, 3:1, 3.5:1 or 4:1, based on the total weight of the composition.

10. The method of claim 1, wherein the ratio of the amount of zinc oxide (by wt %) to zinc citrate (by wt %) is 2:1, based on the total weight of the composition.

11. The method of claim 1 wherein the protein that reduce signals associated with neutrophil recruitment is IL-1Ra, and the proteins that induce neutrophil recruitment are selected from the group consisting of: CXCL10 and G-CSF.
